(19) United States
(12) Patent Application Publication
(10) Pub. No.: US 2026/0224894 A1
(43) Pub. Date: Aug. 6, 2026

(54) DEVICES FOR INTRACAVITY TUMOR TREATING FIELDS

(71) Applicants: The Brigham and Women's Hospital, Boston, MA (US); University of Massachusetts Boston, Boston, MA (US)

(72) Inventors: David Segar, Boston, MA (US); Joshua D. Bernstock, Boston, MA (US); Sumientra Rampersad, Boston, MA (US); David D. Liu, Boston, MA (US)

(21) Appl. No.: 19/150,899

(22) PCT Filed: Jan. 26, 2024
(86) PCT No.: PCT/US2024/013201

§ 371 Date: Jul. 24, 2025

Related U.S. Application Data
(60) Provisional application No. 63/481,787, filed on Jan. 26, 2023.

(51) Int. Cl. A61N 1/36; A61N 1/05

(52) U.S. Cl. CPC A61N 1/36002; A61N 1/05

(57) ABSTRACT

Intracavity devices and methods include and/or utilize a non-conductive core configured to be implanted in a body surface; and a plurality of electrodes disposed on an outer surface of the non-conductive core, wherein the plurality of electrodes are configured to generate a tumor treating field within the body surface.

### CROSS-REFERENCE TO RELATED APPLICATIONS

This application claims the benefit of and priority to U.S. Provisional Patent Application No. 63/481,787, filed on Jan. 26, 2023, entitled “Modeling of Intracranial Tumor Treating Fields for the Treatment of Complex High-grade Gliomas,” the entire contents of which are herein incorporated by reference for all purposes.

### TECHNICAL FIELD

This disclosure relates to the field of tumor treating field therapy, and more particularly to tumor treating field therapy implanted inside the human body, in direct contact or in close proximity to tumor beds.

### BACKGROUND

Non-invasive electric brain stimulation known as tumor treating fields (TTF) offers an approach to combating tumor growth in a myriad of cancers and as such has been described as a fourth treatment modality alongside surgery, chemotherapy and/or radiation.

In one example of TTF, scalp electrodes emit a continuous alternating electric field that creates an intracellular dipole moment during the formation of the mitotic spindle, resulting in a dose-dependent inhibition of tumor growth. As TTF only inhibits tumor growth while in use, efficacy is significantly improved with strict patient compliance (i.e., with the prescribed protocol suggesting that the device be worn for at least 18 hours per day). Of note, both preclinical and clinical evidence suggest that increased field strength within the tumor and increased stimulation time improve TTF efficacy. Accordingly, developing solutions to achieve these clinically relevant parameters is of importance.

One barrier to increasing field strengths via a transcutaneous/calvarial approach is the attenuation of field strength by the skull itself; modeling of transcranial current stimulation and measurements in actual cadavers have shown that up to 75% of injected currents do not reach the brain. Therefore, bypassing the skull is an appealing solution if one is seeking to achieve clinically relevant increases in field strengths within brain tumors. Consistent with this line of thinking, a study utilizing targeted craniectomies in conjunction with TTF to bypass the skull was conceptualized and has shown promising results both in silico and in a phase I clinical trial.

There is a need for systems and methods of TTF therapy that improve therapeutic efficacy, for example via increased field strengths, tumor coverage, time on target (i.e., stimulation hours per day), and the like.

### SUMMARY

The present disclosure overcomes these and other drawbacks by providing systems and methods for neurosurgically implanted intracavity (e.g., intracranial) stimulation solutions that may provide substantial improvements in the treatment of tumors (e.g., HGGs/GBM) by both increasing the strength of the electric fields delivered and the duration of the therapy. The present disclosure provides an initial proof of concept of the benefits of applying intracranial TTF for three clinically challenging cases via the employment of realistic finite element head models. The present disclosure utilizes two measures to analyze the results of the simulations: electric field strength and therapeutic enhancement ratio (TER) in the region of interest (ROI) encompassing the volume of the tumor.

According to one aspect of the present disclosure, an intracavity device is provided. The intracavity device comprises a non-conductive core configured to be implanted in a body surface; and a plurality of electrodes disposed on an outer surface of the non-conductive core, wherein the plurality of electrodes are configured to generate a tumor treating field within the body surface.

According to another aspect of the present disclosure, a method of operating an intracavity device is provided. The method comprises implanting the intracavity device within a body surface, wherein the intracavity device includes a non-conductive core and a plurality of electrodes disposed on an outer surface of the non-conductive core; and generating a tumor treating field within the body surface via the plurality of electrodes.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an example in more detail than is necessary for a fundamental understanding of the teachings of the disclosures. In the drawings:

FIG. 1 illustrates example TER curves in accordance with various aspects of the present disclosure and according to a comparative example.

FIG. 2 illustrates example field strength curves in accordance with various aspects of the present disclosure.

FIG. 3 illustrates computationally optimized intracranial configurations and results in accordance with the present disclosure.

FIG. 4 illustrates example histograms of field strengths within an ROI in accordance with various aspects of the present disclosure.

FIG. 5 illustrates example transcranial results according to a comparative example.

FIG. 6 illustrates example electrode arrays in accordance with various aspects of the present disclosure.

FIG. 7 illustrates example tumor head models in accordance with various aspects of the present disclosure.

FIG. 8 illustrates example electrode configurations according to a comparative example.

FIG. 9 illustrates an example electrode configuration according to various aspects of the present disclosure.

FIG. 10 illustrates an example fit of in vitro TER data in accordance with various aspects of the present disclosure.

FIG. 11 illustrates an example electrode numbering in accordance with various aspects of the present disclosure.

FIG. 12 illustrates an example electrode and core array in accordance with various aspects of the present disclosure.

FIG. 13 illustrates an example device with field representation in accordance with various aspects of the present disclosure.

FIG. 14 illustrates an example of an externally-connected electrode array in accordance with various aspects of the present disclosure.

FIG. 15 illustrates an example of an electrode array in an intracranial location in accordance with various aspects of the present disclosure.

FIG. 16 illustrates an example of a scaffolding arrangement in accordance with various aspects of the present disclosure.

FIG. 17 illustrates an example of a cooling device in accordance with various aspects of the present disclosure.

FIG. 18 illustrates an example of a cooling device in accordance with various aspects of the present disclosure.

### DETAILED DESCRIPTION

In the following detailed description, reference is made to the accompanying drawings in which specific examples are shown by way of illustration. These examples are described in sufficient detail to enable those of ordinary skill in the art to practice the disclosure. It should be understood, however, that the detailed description and the specific examples, while indicating examples of embodiments of the disclosure, are given by way of illustration only and not by way of limitation. From this disclosure, various substitutions, modifications, additions rearrangements, or combinations thereof within the scope of the disclosure may be made and will become apparent to those of ordinary skill in the art.

Unless otherwise indicated, the various features illustrated in the drawings may not be drawn to scale. The illustrations presented herein are not necessarily intended to be actual views of any particular method, device, or system, but are merely idealized representations that are employed to describe various embodiments of the disclosure. Accordingly, the dimensions of the various features as illustrated may be arbitrarily expanded or reduced for clarity. In addition, some of the drawings may be simplified for clarity. Thus, the drawings may not depict all of the components of a given apparatus (e.g., device) or method. In addition, like reference numerals may be used to denote like features throughout the specification and figures.

It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. Also, unless stated otherwise a set of elements may comprise one or more elements.

Unless otherwise specified or indicated by context, the terms “a,” “an,” and “the” mean “one or more.” As used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “only one of,” or “exactly one of.” For example, a list of “only one of A, B, or C” indicates options of: A, but not B and C; B, but not A and C; and C, but not A and B. In contrast, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more A, one or more B, and one or more C. Similarly, a list preceded by “a plurality of” (and variations thereon) and including “or” to separate listed elements indicates options of one or more of each of multiple of the listed elements. For example, the phrases “a plurality of A, B, or C” and “two or more of A, B, or C” indicate options of: one or more A and one or more B; one or more B and one or more C; one or more A and one or more C; and one or more A, one or more B, and one or more C.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the term “subject” may be used interchangeably with the term “patient” or “individual” and may include an “animal” and in particular a “mammal.” Mammalian subjects may include humans and other primates, domestic animals, farm animals, and companion animals such as dogs, cats, guinea pigs, rabbits, rats, mice, horses, cattle, cows, and the like.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

As will be described herein, the present disclosure provides systems and methods for intracavity TTF. Several examples are provided directed to intracranial TTF; however, these examples are not limited and solely presented by way of illustration and explanation. In practice, the present disclosure is not limited to intracranial treatment modalities, and may be applied to other modalities such as the abdominal cavity, the chest cavity, the neck, soft tissues and bone- and neoplasms associated with those parts of the body.

In view of the above-noted deficiencies and challenges in comparative systems and methods of TTF, it was hypothesized that neurosurgically implanted intracranial stimulation system(s) may provide substantial improvements in the treatment of HGGs/GBM by both increasing the strength of the electric fields delivered and the duration of the therapy. The present disclosure provides a proof of concept of the benefits and challenges of optimizing intracranial TTF for three clinically challenging cases via the employment of realistic finite element head models. The present disclosure uses two measures to analyze the results of the simulations: electric field strength and therapeutic enhancement ratio (TER) in the region of interest (ROI) encompassing the volume of the tumor.

Using comparative methods, increasing the intensity of tumor treating fields (TTF) within a tumor bed improves clinical efficacy, but reaching sufficiently high field intensities to achieve growth arrest remains challenging due in part to the insulating nature of the cranium. By contrast, using MRI-derived finite element models (FEMs) and simulations, the present disclosure provides a set of intracranial electrode locations to obtain increased TTF intensities in three clinically challenging high-grade glioma (HGG) cases (i.e., thalamic, left temporal, brainstem). Electric field strengths were converted into therapeutic enhancement ratios (TER) to evaluate the predicted impact of stimulation on tumor growth. Concurrently, conventional transcranial configurations were simulated/optimized for comparison. Intracranial TTF were able to achieve field strengths that have been shown capable of inducing complete growth arrest in 98-100% of the tumor volumes using only 0.54-0.64 A current. The reconceptualization of TTF as a targeted, intracranial therapy has the potential to provide a meaningful survival benefit to patients with HGG and other brain tumors, including those in surgically challenging, deep, or anatomically eloquent locations which may preclude surgical resection.

In the brain, the disclosed system and method help solve a number of practical/patient compliance issues (e.g., via the elimination of the need for daily head shaving and large electrode patches covering the head). It is prudent to note that TTFs only inhibit tumor growth while in use and that efficacy is significantly improved with patient compliance (i.e., with prescribed protocol suggesting that the device be worn for at least 18 hours per day).

Systems in accordance with the present disclosure may have a non-conductive core forcing current to flow through the surrounding peri-tumoral tissue. The non-conductive core can be coated in electrodes; current source and current sink would be located on distinct surfaces of the device with the goal of forcing current through the diseased/at risk peri-resectional tissue. The conceptualized intervention is distinguished, at least in part by a design which encompasses the volume of a resection cavity or other hollow body cavity with the purpose of disturbing TTF locally and continuously in a focused manner. Several different examples of this structure have been developed including but not limited to the following: electrodes surrounding an expansile balloon-like non-conductive core; a 3D printed device customized to a patient's resection cavity coated in electrodes; pre-sized electrode cages for generic/off the shelf-use; and/or an electrode array support by interior scaffold (custom vs off-the shelf/generic).

Comparative examples of tumor treating fields electrodes may be placed on the body surface allowing for heat dissipation into the external environment. In an implanted device, such as those described herein, heat generation would be significant given the high levels of electrical energy transmitted by the device. In order to combat heat generation in such a scenario, in some examples the present disclosure is embodied in a cooled device which may result in several examples described below. In the first example, a liquid coolant may be passed through the hollow core of the device and after circulating would be transmitted to a heat sink (which itself may be implanted in the body and/or be external). In one variation of this device the heat sink could be large vasculature within the body. In another example, the electrodes may be juxtaposed to a Peltier junction (i.e., with the cooling surface paired next to the electrode and the heated surface paired to coolant that would be transmitted to a separate heat sink). Such a device may be used in the care of brain tumor patients, and would also have a role in other solid tumors throughout the body that have been resected but that still require adjuvant chemotherapy and/or radiation. Finally, such a device may also be capable of recording from the brain in its final form and/or when the frequency of the field is adjusted help control intractable infections.

In support of the efficacy of systems and methods in accordance with the present disclosure, 17 transcranial and 5050 intracranial configurations were analyzed to identify those that required minimum current to achieve TER >0 or TER >1 within the ROIs for the three tumor cases presented. The TER quantifies the effects of an electric field on a tumor: TER values above 0 indicate a reduction in tumor growth, TER of 1 corresponds with complete growth arrest, and TER values above 1 indicate tumor shrinkage. Complete proliferation arrest (TER of 1) occurs in glioma cell cultures when fields reach 2.25 V/cm in the treated tumor. Therefore, the percentage of the ROI receiving field strengths over 2.25 V/cm and the percentage for which TER>1 were calculated as primary outcome measures. Since it may be expected that lower field strengths (above 1.10 V/cm) also have an inhibitory effect on tumor growth, results for field strengths above 1.50 V/cm and TER >0 are additionally provided.

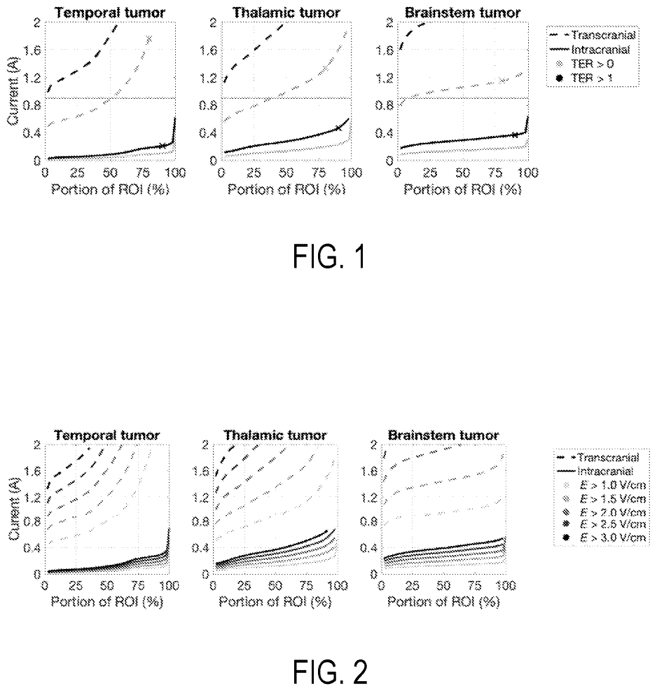

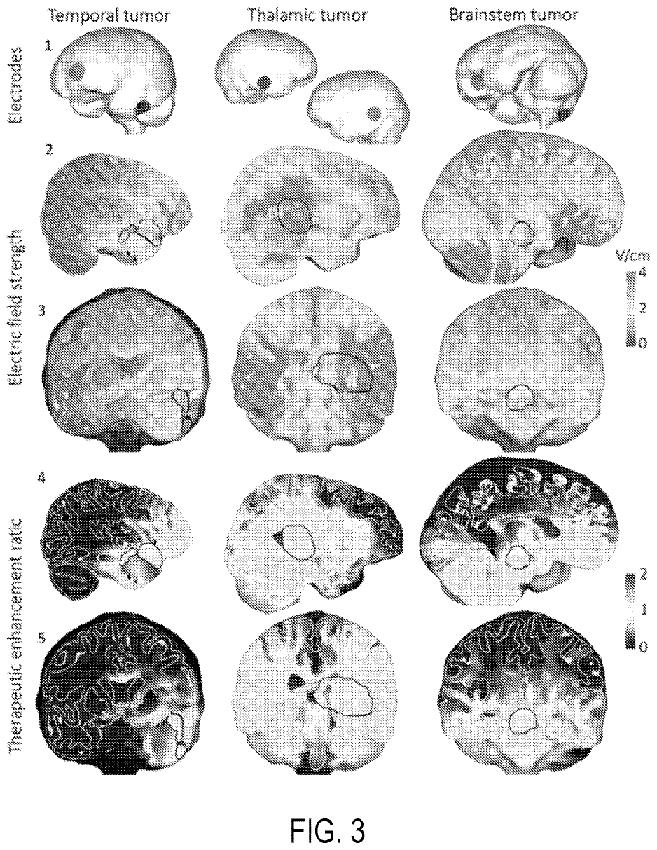

FIG. 1 illustrates curves that display the minimum required current for various percentages of ROI volume with TER >0 or TER >1. Each curve indicates the amount of current needed to achieve a TER greater than 0 (gray) or 1 (black) in a percentage of the ROI for transcranial (dashed) and intracranial (solid) TTF for three tumor cases. Horizontal lines indicate the current level used for transcranial TTF according to a comparative example (0.9 A). Crosses indicate configurations that were selected, which are visualized in FIGS. 2 and 3.

FIG. 2 shows similar curves for field strength and Table 1 below presents statistics. In FIG. 2, each curve indicates the amount of current needed to achieve a certain electric field strength (E) in a given percentage of the ROI volume. For example, the first panel represents the results for the temporal tumor; the dashed black line (top most line) shows that with a transcranial configuration, one can reach a field strength of at least 3.0 V/cm in 25% of the ROI volume using 1.8 A of current. Horizontal lines indicate the current level used in comparative examples for transcranial TTF (0.9 A). The amount of current needed to achieve a certain field strength in a given percentage of the ROI increases linearly with the desired field strength. For the brainstem tumor (panel 3), 3.0 V/cm is not reached in any part of the ROI, hence there is no corresponding curve. In Table 1, the top half of the table presents, for each of the three tumor cases, the percentage of ROI volume in which a certain TER value can be achieved given a desired amount of injected current, using either comparative (transcranial) or intracranial configurations. The bottom half of the table presents the minimum amount of current needed to achieve a certain TER or field strength in a desired percentage of the ROI volume.

TABLE 1

| Result | Method | Temporal | | Thalamic | | Brainstem | |
|---|---|---|---|---|---|---|---|
| Portion of ROI reaching TER > 0 | Transcranial | 52% | | 40% | | 8% | |
| with 0.9 A injected current (amount | Intracranial | 100% | | 100% | | 100% | |
| for comparative transcranial TTF) | | | | | | | |
| Portion of ROI reaching TER > 1 | Transcranial | 2% | | 2% | | 2% | |
| with 0.9 A injected current | Intracranial | 100% | | 98% | | 100% | |
| Portion of ROI reaching TER > 0 | Transcranial | 82% | | 96% | | 98% | |
| with 2 A injected current | Intracranial | 100% | | 100% | | 100% | |
| (maximum amount simulated) | | | | | | | |
| Portion of ROI reaching TER > 1 | Transcranial | 56% | | 48% | | 24% | |
| with 2 A injected current | Intracranial | 100% | | 100% | | 100% | |
| Current needed to achieve TER > 0 | Transcranial | 0.89 | A | 0.99 | A | 1.06 | A |
| in 50% of the ROI | Intracranial | 0.04 | A | 0.14 | A | 0.14 | A |
| Current needed to achieve TER > 0 | Transcranial | 0.31 | A | 0.58 | A | 0.32 | A |
| in 100% of the ROI | | | | | | | |
| Current needed to achieve TER > 1 | Intracranial | 0.62 | A | 0.54 | A (98%) | 0.64 | A |
| in 100% of the ROI | | | | | | | |
| Current needed to achieve E > [1.0, | Transcranial | 0.81 | A | 0.9 | A | 0.96 | A |
| 1.5, 2.0, 2.5, 3.0] V/m in 50% of the | | 1.21 | A | 1.35 | A | 1.44 | A |
| ROI | | 1.62 | A | 1.8 | A | 1.92 | A |
| | Intracranial | 0.04 | A | 0.12 | A | 0.13 | A |
| | | 0.06 | A | 0.18 | A | 0.2 | A |
| | | 0.08 | A | 0.25 | A | 0.26 | A |
| | | 0.1 | A | 0.31 | A | 0.33 | A |
| | | 0.12 | A | 0.37 | A | 0.39 | A |

Transcranial TTFs were the most effective (i.e., highest percentage reached) for the temporal and least effective for the brainstem tumor, while intracranial TTFs were the least effective for the thalamic tumor. Using 0.9 A current (the clinically employed value for comparative TTF), transcranial TTFs reached TER >1 in 2% of the ROI volumes examined and TER >0 in 8-53% across al ROIs while intracranial TTFs achieved TER >1 in 98-100%, and TER >0 in 100% of the ROIs for all three tumor cases. Interestingly, achieving TER >0 in 50% of the ROIs examined required 7-15 times as much current with transcranial as compared to intracranial TTFs.

To examine the electric field and TER distributions throughout the brain, we selected one “preferred” configuration for each case; this was defined as the configuration that required the minimum current to achieve TER >1 in at least 90% of the ROIs (marked with a cross in FIG. 1); such configurations may be expected to have high clinical efficacy without needing to maximize the injected current. The preferred intracranial configuration for the left temporal tumor consisted of one electrode along the left anterior orbitofrontal cortex and one electrode on the posterior left temporal lobe (FIG. 3, top row). Of note, electrodes close to the ROI and on opposite sides appear to produce the largest electric fields in a superficial ROI. The preferred configuration for the thalamic tumor consisted of one electrode on left parietal cortex and one on right lateral cerebellum abutting the petrous face, resulting in current that flowed roughly through the center of the brain (FIG. 3, top row). These electrodes were farther away from the ROI than for the temporal tumor and were in both hemispheres, thereby directing the current to a central ROI. The preferred brainstem configuration consisted of one electrode on left orbitofrontal cortex and one on right posteroinferior cerebellum above the foramen magnum, resulting in an anteroposterior current flow on the inferior brain surface (FIG. 3, top row).

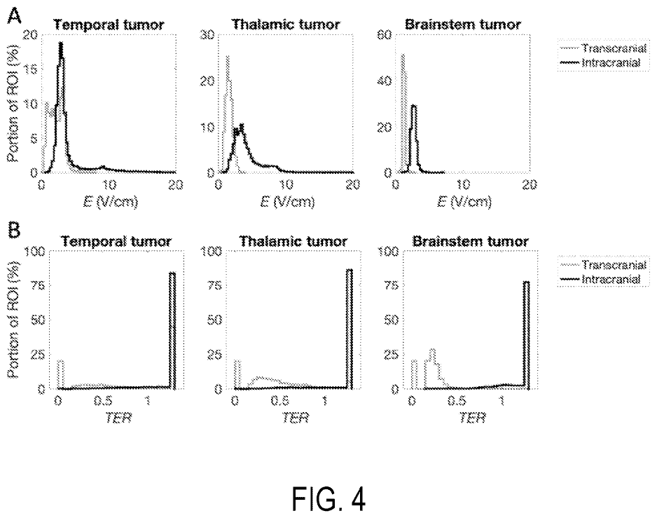

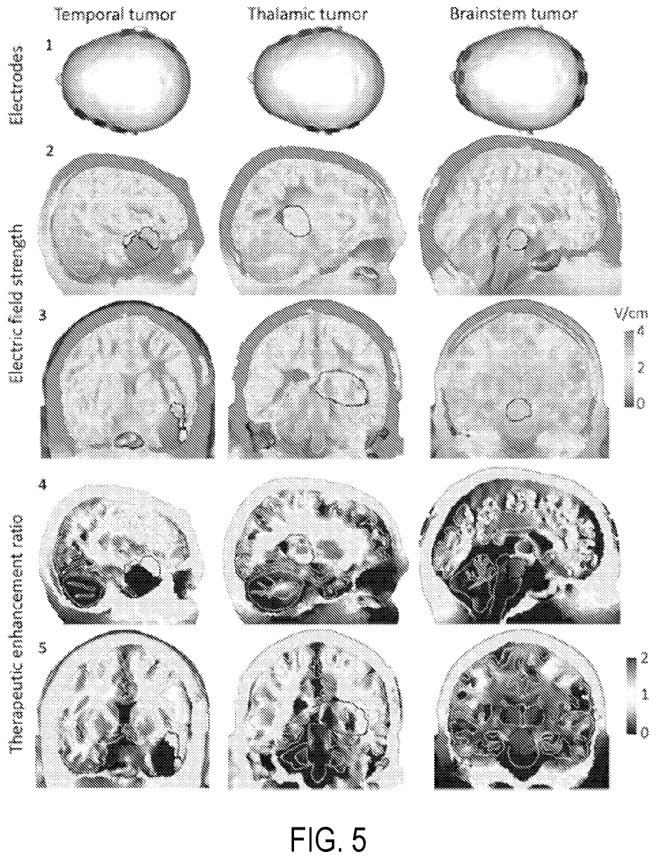

The electric field in the brain was much more focal for the temporal tumor than for the other two cases (FIG. 3, rows 2-3) due to the ROI being relatively superficial, resulting in electrodes that were in close proximity. Results are shown on cuts through the center of the tumor, which is outlined in black. Gray and white matter boundaries are shown in white. Histograms of field strengths within the ROI are shown in FIG. 4. Critically, as compared to the preferred transcranial results (FIG. 5), the intracranial models produced stronger and more focal fields. For FIG. 4, preferred configurations were defined as those that achieve TER >0 for transcranial TTF, or TER >1 for intracranial TTF, in at least 90% of the tumor volume with the minimum amount of current needed to achieve this. These configurations are marked with a cross in FIG. 1, which will be described in more detail below.

Rows 4-5 of FIG. 3 show TER on the same cross planes: all yellow areas reached TER >1 and as such would be expected to achieve proliferation arrest and/or tumor shrinkage in clinical practice. Using the intracranial configurations this was achieved in majority of the tumor ROIs for each of the three cases examined. While the thalamic tumor core was projected to receive lower field strengths than the shell/surrounding tissue due in part to the core's higher conductivity, most of the core did reach clinically relevant TER >0. Histograms of TER values within the ROIs for the intracranial configurations were similar with large peaks having been noted for TER=1 (see FIG. 4, set B). As evidenced by the distributions in the temporal and thalamic and brainstem ROIs, transcranial stimulation was much less effective at achieving clinically relevant TERs (see FIG. 4, set B).

In FIG. 4, set A illustrates histograms of electric field strength in the ROI. For intracranial stimulation, histograms peak around 3.0 V/cm for all cases, but the distributions in the temporal and brainstem ROIs are fairly narrow, while the thalamic ROI receives higher field strengths in a large portion of the ROI. With transcranial stimulation, peaks occur at lower field strengths. There is more overlap with the intracranial distribution for the temporal ROI, because it is more superficial and easier to reach transcranially compared to the other two. Set B illustrates histograms of therapeutic enhancement ratio (TER) in the ROI. For intracranial stimulation, all distributions have a large peak at TER=1, with a bump at slightly lower TER for the brainstem ROI. For transcranial stimulation, there is a much smaller peak at TER=1 for the temporal and thalamic ROIs, while the brainstem ROI does not achieve TER=1 anywhere. Each case also has a peak at TER=0 and a bump at slightly higher values.

FIG. 5 illustrates the preferred transcranial configurations and results. The preferred configurations (row 1) produced electric fields in the brain (rows 2-3: sagittal and coronal views) that achieved a therapeutic enhancement ratio (TER) in the tumor (rows 4-5: identical views). The cuts are identical to those used in FIG. 3. The selected configurations achieved TER >0 in at least 90% of the ROI (modeled tumor) with the minimum amount of current needed to achieve this. These configurations are marked with a gray cross in FIG. 1.

When providing a single “preferred” configuration, it is to be noted that one of its electrode locations may in fact be difficult to reach surgically. The benefit of the approach described here is that it also provides multiple “near-optimal solutions” as described in Table 2. The differences in current required between the first and fifth solutions are 0.9-3.8% across the three cases, demonstrating that multiple configurations can essentially achieve the same effects with similar amounts of current. For the temporal and thalamic cases, each of the top five configurations has one electrode in common, while for the brainstem case, there is more variety. If for a specific patient, one of these selected electrode locations could not be reached surgically, one could ultimately remove that electrode from the set and repeat the optimization.

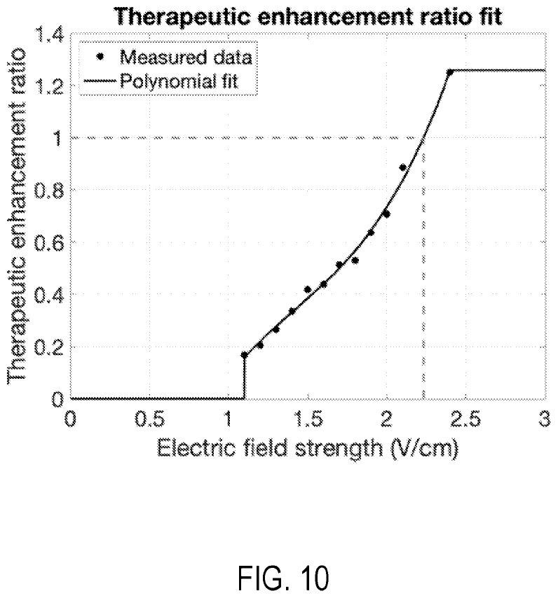

Table 2 illustrates the five best configurations for intracranial TTF for the three tumor cases. For each case, the table presents the top five configurations that require the least amount of current to reach a therapeutic enhancement ratio (TER) greater than 1 in at least 90% of the ROI (the modeled tumor). For each configuration (each row), the table presents the required current amplitude, the percentage of the ROI that reaches TER >1, and the numbers of the electrodes that make up that configuration. Electrode numbers are visualized on the model in FIG. 10, described in more detail below.

TABLE 2

| | Temporal Tumor | | | Thalamic Tumor | | | Brainstem Tumor | | |
|---|---|---|---|---|---|---|---|---|---|
| | | TER > 1 | elec- | | TER > 1 | elec- | | TER > 1 | elec- |
| | I(A) | (%) | trodes | I(A) | (%) | trodes | I(A) | (%) | trodes |
| 1 | 0.206 | 90.3 | 1, 84 | 0.469 | 90.0 | 46, 67 | 0.369 | 90.3 | 82, 99 |
| 2 | 0.206 | 90.2 | 27, 84 | 0.472 | 90.1 | 46, 79 | 0.378 | 90.2 | 82, 96 |
| 3 | 0.209 | 90.3 | 4, 84 | 0.472 | 90.0 | 46, 93 | 0.380 | 90.4 | 90, 91 |
| 4 | 0.213 | 90.3 | 62, 84 | 0.472 | 90.0 | 46, 94 | 0.382 | 90.1 | 91, 99 |
| 5 | 0.217 | 90.0 | 31, 84 | 0.473 | 90.1 | 12, 46 | 0.383 | 90.2 | 73, 82 |

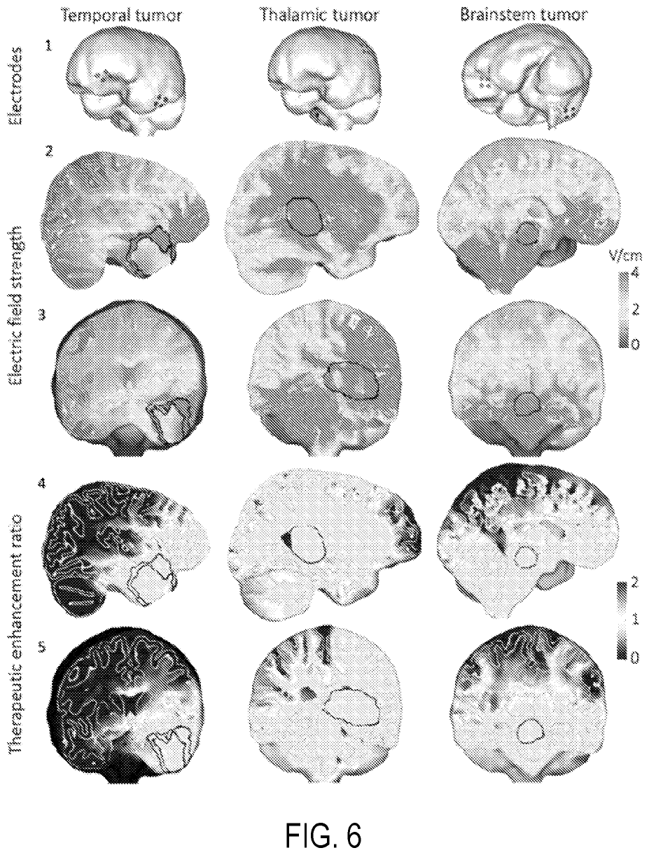

As discussed above electrodes were modeled as 10-mm disks, based on the comparative example Novocure TTF array. Intracranial cortical stimulation for other purposes may be done using grids of small circular electrodes. Using an array of smaller electrodes will affect the resistance of the system and therefore can change the intensity of the fields. To investigate these effects, we replaced the electrodes from FIG. 3 with 2×2 arrays of 3-mm radius electrodes (FIG. 6) and simulated TTF with the same amount of current spread across the electrodes of each array. In particular, FIG. 6 shows an example in which the preferred electrode configurations shown in FIG. 3 were replaced with 2×2 arrays of small electrodes (row 1) and simulated with the same amounts of current (see Table 2). This produced electric fields in the brain (rows 2-3: sagittal and coronal views) that achieved a therapeutic enhancement ratio in the tumor (rows 4-5: identical views). The cuts are identical to FIG. 3. The resulting distributions of electric field strength and TER throughout the brain strongly resemble those for stimulation with single large electrodes (FIG. 6 vs FIG. 3), however, field strengths are higher when using the 2×2 arrays, as was the percentage of tumor volume receiving TER >1.

Evidence exists to suggest that higher electric field strengths result in more effective inhibition of tumor growth with resultant improvements in overall survival in GBM. While the dose response curves used to estimate rates of tumor inhibition rely on in vitro data, it is accepted that field intensity predicts the degree of tumor inhibition as a measure of treatment dose. For example, higher field strengths correspond with longer median survival. A second component of treatment dose is the amount of time that the therapy is delivered, and extending daily treatment duration contributes to improved survival. The present disclosure demonstrates the potential for substantial improvement in the TTF dose using an implanted stimulator by improving both strength and duration of TTF delivery.

Due to anatomic and biophysical constraints, transcranial stimulation with Novocure's comparative Optune (R) device provides a variable degree of therapeutic field intensity to treatment area(s) within the brain, but as evidenced above is generally unable to reach doses associated with complete tumor arrest in large portions of the tumor volume. It is also to be noted that unlike radiation, which may affect the entire tumor volume, TTFs are likely to have highly heterogeneous field strengths within the tumor due to variations in conductivity, proximity to CSF spaces, electrode location and/or anatomic variations such as skull thickness and tumor depth.

Tumor and treatment heterogeneity have implications and conceptually, the inability to reach therapeutic intensities within an entire tumor bed is in some ways functionally akin to a subtotal resection, with a larger untreated percentage of tumor conferring worse clinical outcomes. Surgery remains used in GBM therapy, and gross total resection confers a survival advantage during initial surgery and at the time of recurrence. A similar phenomenon may logically exist in TTF, such that treating nearly 100% of the tumor volume or peri-resection region with high intensity fields may confer a significant survival benefit. Accordingly, the present disclosure supports that using intracranial electrode arrays may provide a modality to accomplish such a goal.

As surgery is already part of the of care for GBM, neurosurgeons are heavily involved in the care of such tumor patients. In addition to resective surgeries, several experimental immunotherapies rely on neurosurgical delivery of the therapeutic agent to the tumor bed. The addition of another surgical modality into the therapeutic arsenal targeting GBM would therefore likely be widely accepted by practitioners in both neurosurgery and neuro-oncology.

Certain cases, such as thalamic or brainstem GBMs, are often unresectable due to the involvement of eloquent and/or critical brain structures. In such cases, prognosis is even worse, with median survivals of only 12.2 months having been documented in patients with thalamic GBM, 6 months for brainstem GBM, and approximately 12 months for pediatric diffuse intrinsic pontine glioma (DIPG). As demonstrated above, despite attempts at optimization of electrode placement and skull reduction surgery, tumors located deep within the brain remain elusive targets for transcranial TTFs. Such cases are among the examples of where intracranial TTF may be of immediate clinical benefit. Despite the potential of comparative transcranial TTF therapy, adoption remains infrequent among patients with GBM and their treating physicians. In addition to the critiques over trial design, others have emphasized a “hassle” factor due to frequent head shaving, daily device placement, and the visibility of a device associated with brain cancer treatment, which may result in decreased quality of life. Concerns about patient convenience and compliance were considered substantial barriers to use in a recent survey of medical and neurooncologists. When paired with what has been considered a relatively modest increase in overall survival, these obstacles have proved difficult to surmount. An intracranial approach as described herein may overcome several of these barriers; with an implanted electrode array, patients would no longer need to shave or apply large electrode pads to the scalp. In addition, a fully implanted system could function similarly to a deep brain stimulation system and run up to 24 hours per day, if desired, all of which are expected to result in improved compliance and ultimately clinical outcomes. Such an approach may also serve an adjuvant treatment modality for to a litany of other emerging experimental therapies (e.g., immunotherapy). Lastly, an implanted array may drive innovation in the growing field of cancer neuroscience, for example creating a platform that may enable physiologic recording within and/or adjacent to a tumor

The comparative Optune device delivers current with an amplitude of up to 0.9 A but not all of it reaches the brain given diffusion into the scalp and the insulating effects of the skull. If a 75% loss is assumed, the current reaching the brain is 2.4-2.8 times lower than the 0.54-0.64 A needed for the intracranial models to achieve growth arrest in the complete tumor, or approximately 0.225 A. If 0.225 A is injected intracranially, growth arrest can be achieved in 14-96%, and growth reduction in 90-100%, of the ROIs, as compared to 2% and 8-52% with 0.9 A transcranial TTF. This approximation offers evidence that the stimulation parameters proposed herein compare favorably with the stimulation intensity delivered via approved treatment methods.

The reduced energy requirement for the use of an intracranial array may be provided by implantable medical batteries or by the use of an external power supply with an implanted device, as is used with ventricular assist devices. Additionally, cells are more affected by TTF when mitotic spindles are directionally aligned with the electric fields, and the interleaved application of fields in multiple directions has been shown to increase the strength of tumor inhibition in both cell culture and animal models. In support of the present disclosure, all possible configurations of intracranial electrode pairs and a comprehensive set of transcranial pairs were evaluated. When designing a clinical array, a set of two roughly perpendicular pairs could be selected from all modeled pairs using the analysis methods described here.

The reconceptualization of TTF as a targeted, invasive therapy has the potential to provide a meaningful survival benefit to all patients with GBM and other brain tumors, even those with unresectable or surgically challenging tumors. The combination of in vitro dose response curves, clinical data corroborating the impact of higher TTF doses on improved survival, and the in silico evidence presented here supports the use of an intracranial TTF stimulator. Research in TTF will benefit tremendously from an ongoing multi-disciplinary approach; neuro-oncologists and surgeons should work closely with engineers and computer scientists to optimize the therapy and hopefully substantially improve outcomes for patients with GBM.

### Examples—Materials and Methods

Via the finite element modeling (FEM) of intracranial versus transcranial electrodes, TTFs were investigated for three surgically complex GBM cases. Tumors were segmented from patient scans and built into a detailed base head model to allow for comparison between cases without the confounding effects of individual head geometry. Electrodes were built into the models' skin (transcranial) or CSF (intracranial) surface and simulations were performed for a comprehensive set of electrode configurations and current amplitudes. Finally, optimal stimulation parameters were selected for transcranial and intracranial TTFs and in so doing the theoretical treatment outcomes were compared.

### Head Models

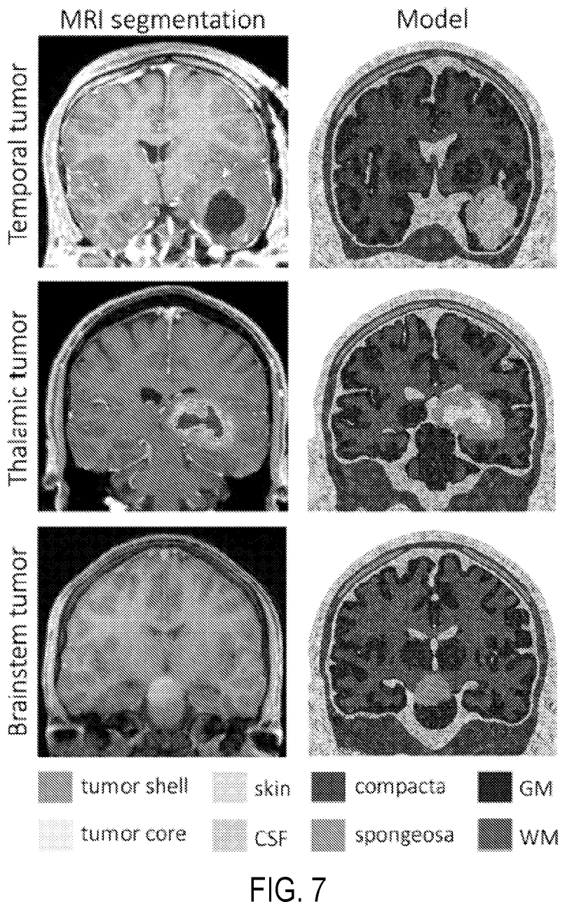

A finite element model was generated from T1-, T2- and diffusion-weighted MR images of a healthy 25-year-old man, as previously described. Conductivity tensors were calculated using the volume-normalized approach and multiplied with the effective conductivity values for GM (0.276 S/m) and WM (0.126 S/m). All other compartments were assigned isotropic conductivities: skin (0.465 S/m), skull compact bone (0.007 S/m), skull spongy bone (0.025 S/m), CSF (1.65 S/m), eye (1.5 S/m), muscle (0.4 S/m). For simulations of intracranial TTFs, tissue compartments outside of the CSF were removed for ease of modeling, as has been done in electrocorticography simulations. Removing these superficial tissue layers may result in a slight overestimation of the intracranial field strengths; however, in practice, these differences are expected to be minimal, given the presence of a silicone insulating layer on most intracranial electrodes, and the substantially lower conductivity of skull when compared to CSF and brain tissues. Three representative HGG cases treated at Brigham and Women's Hospital between 2018 and 2020 were selected; informed consented was acquired. Evaluation of patient data was approved by the Mass General Brigham Institutional Review Board (IRB); data/experiments were collected/conducted in accordance with pertinent National Institutes of Health (NIH) guidelines. The three representative tumors selected were as follows: a left temporal GBM status post subtotal resection, a thalamic GBM, and an isolated HGG within the brainstem. T1 images of each patient and the base head model were skull stripped using the FSL bet program, followed by registration of patient scans to the model scan using a linear transform in 3DSlicer. The tumor shell (all cases), core (thalamic tumor) and resection area (temporal tumor) were segmented from the registered scans in Seg3D (FIG. 7, column 1). The segmentation masks were then mapped onto the base model using SCIRun (FIG. 7, column 2). Thus, in FIG. 7, for the three tumor cases, tumors were segmented from patient MRIs (left) and the segmentation masks (outlines on the MRIs) were mapped onto the base model, resulting in three head models (right). The tumor core (1.0 S/m) and shell (0.24 S/m) were given different conductivities and the resected area was assigned the conductivity of CSF.

### Electrode Configurations

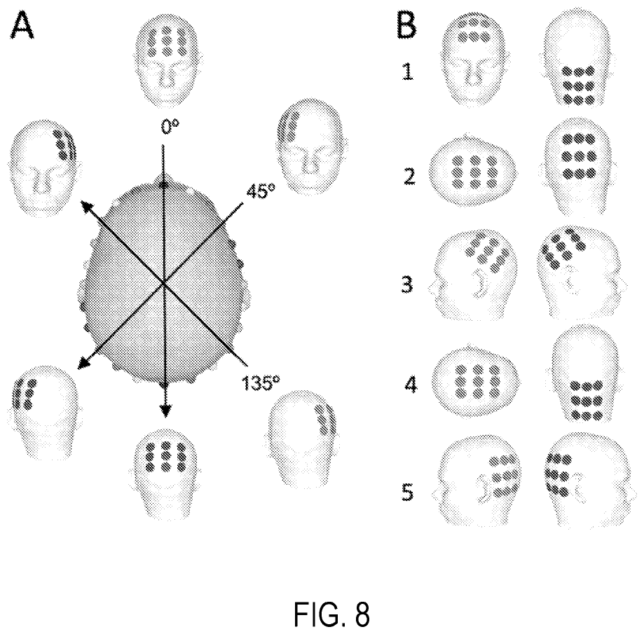

Transcranial: A Novocure 3×3 electrode patch with 22 (vertical) and 33 (horizontal) mm respectively between electrode centers was modeled, with an electrode radius of 10 mm and height of 2.5 mm. Electrodes were placed on the skin surface in an anteroposterior configuration similar to Novocure's proscribed recommendations. FIG. 8 illustrates the transcranial electrode design. In set A, the center image shows the transcranial model from above. Beginning with the line marked 0°, two patches were centered on the locations indicated by the blue spheres, resulting in the model shown at the top (anterior view) and bottom (posterior view) of the line. This pair of patches was then rotated around the vertical axis; spheres on the center image indicate the patch centers. Examples are shown at 0°, 45°, and 135°. Set B illustrates five transcranial configurations that were based on other studies. The pair of patches was rotated around the vertical axis of the head in 15-degree steps, ultimately producing 12 configurations (see, e.g., FIG. 8, set A). Configurations from prior modeling studies were recreated (see FIG. 8, set B): 1) anteroposterior with lower posterior electrode, 2) patches on the top and back of the head, 3) patches above the ears; and two configurations proposed for infratentorial stimulation by Novocure: 4) patches on the top of the head and the neck, 5) patches behind the ears. The complete set comprised 17 models in total.

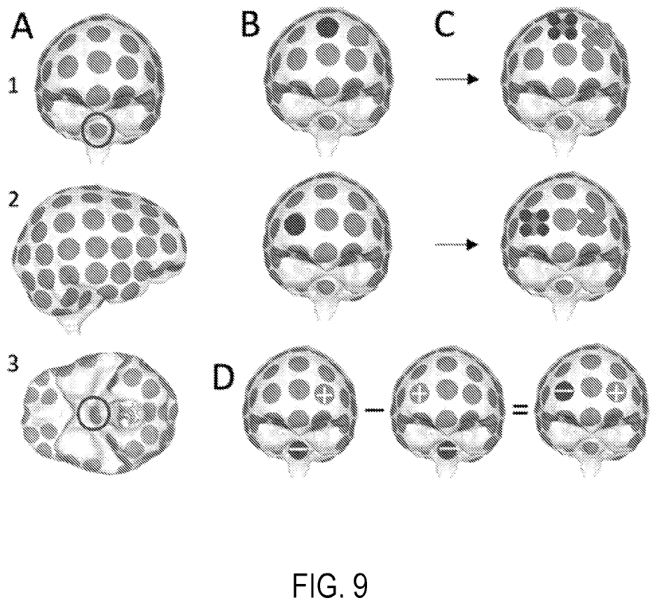

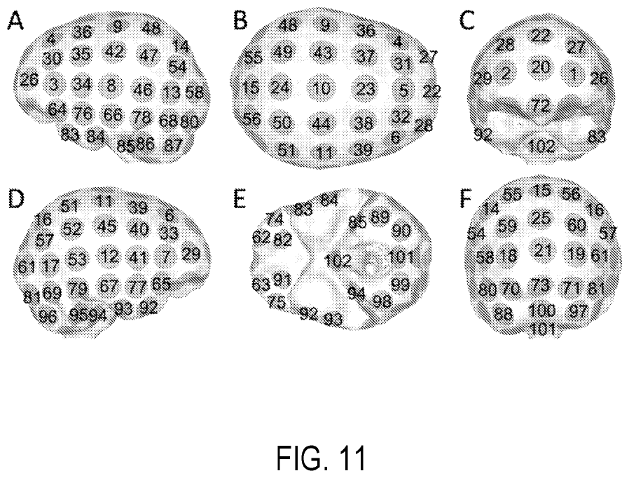

Intracranial: Pairs of 10-mm electrodes were used as placeholders to allow for an examination of the effects of different locations without assumptions with regard to size and/or numbers. FIG. 9 illustrates examples of intracranial electrode configurations. In set A, anterior (1), right (2), and inferior (3) views of the intracranial model are presented, where 101 electrodes were spread across the CSF surface and a reference (for purposes of simulation, but not necessary in clinical practice) was placed on the brainstem (marked with blue circles). FIG. 11 illustrates additional views. In set B, two examples of 2-electrode configurations are shown. In set C, an example of small-electrode arrays are shown. In Set D, electric fields for each electrode pair were calculated by simulating each electrode with the reference (first two images), and then the difference of the two was taken (result on the right).

Sixty-one electrodes were placed on the model's CSF surface based on the 10-10 electrode systems, and 40 electrodes were added below (see FIG. 9, set A). Electrodes were not placed along the interhemispheric space or abutting the tentorium given that these locations are difficult to reach surgically. One reference electrode was placed in an area not intended for stimulation. There are

$$\frac{101 !}{2 ! ( 101 - 2 ) !} = 5050$$

ways to take 2 out of 101 electrodes, each of which was simulated (see two examples in FIG. 9, set B). Of note, once the locations are identified, smaller electrodes can replace larger ones if so desired (see two examples in FIG. 9, set C).

### Simulations

For each transcranial configuration, the quasistatic approximation to Maxwell's equations was solved using SCIRun 4.7. Intracranial configurations were simulated by first solving for each of 101 electrodes paired with the reference, and then taking linear combinations of the resulting electric fields (example in FIG. 9, set D).

### Predicted Clinical Efficacy

As discussed above, the therapeutic enhancement ratio (TER) quantifies how electric field strength (E) affects tumor growth with reported TER values for tumor cell cultures resulting from the application of electric fields of various strengths. A third-degree polynomial was fitted to the in vitro data: TER (E)=0.4057E³-1.713E²+2.941E-1.542 (see FIG. 10). Data was available for E values from 1.10 to 2.40 V/cm, so TER was set for E lower than 1.10 V/cm to 0, and for E higher than 2.40 V/cm to the value at E=2.40 V/cm: TER (2.40)=1.26 (FIG. 10). In FIG. 10, black dots are a reproduction of reference data, which applied TTF to malignant glioma cell cultures and measured TER values. A polynomial (black line) was fit to the experimental data and values were capped outside of the reported range. For each simulated electric field, this function was used to calculate TER in each element of the model. TER values in the tumor were then used to find electrode configurations that maximized the tumor volume for which TER >0, which corresponds to reduced tumor growth, or TER >1, which corresponds to growth arrest. As can be seen from FIG. 10, complete growth arrest happens at E=2.23 V/cm (dashed line).

### Analysis

A pareto analysis was performed that balanced the injected current and TER achieved in the regions of interest (ROI) within the models. The ROI was defined as all tumor tissue, which for the thalamic tumor included the core and shell. For each configuration, electric fields were calculated for 2000 values of injected current linearly distributed between 0 and 2 A. For each solution, the percentage of ROI volume for which the TER >0, or TER >1, was calculated. The solutions were then grouped based on their percentage into bins between 0% and 100%; in each bin, the configuration that required the least amount of current to achieve that percentage was selected. Because lower field strengths may still have some therapeutic relevant effects, and higher field strengths are expected to have a greater effect, this analysis was repeated for field strengths between 1.0 and 3.0 V/cm.

In the above example, and particularly in Table 2, certain electrodes are referred to according to a numbering scheme. FIG. 11 illustrates the electrode numbering used for the intracranial model. Left (A), top (B), anterior (C), right (D), bottom (E) and posterior (F) views of the model's outer surface (i.e., the CSF surface) with electrodes embedded are shown. These numbers correspond to the configurations listed in Table 2.

### Example Implementations

While the above simulations and discussions were presented from the perspective of brain surface electrode arrays, the present disclosure is not so limited. In addition to brain surface electrode arrays, the present disclosure may be realized through the use of intraparenchymal depth electrodes, intra-cavitary electrode arrays, or a combination of different electrode techniques, to obtain a maximally efficient coverage of tumor volumes. Depending on the implementation, the use of intraparenchymal depth electrodes and/or intra-cavitary electrode arrays may further increase field strengths within a tumor and reduce power requirements by shortening the proximity between the treatment volume and electrode contacts.

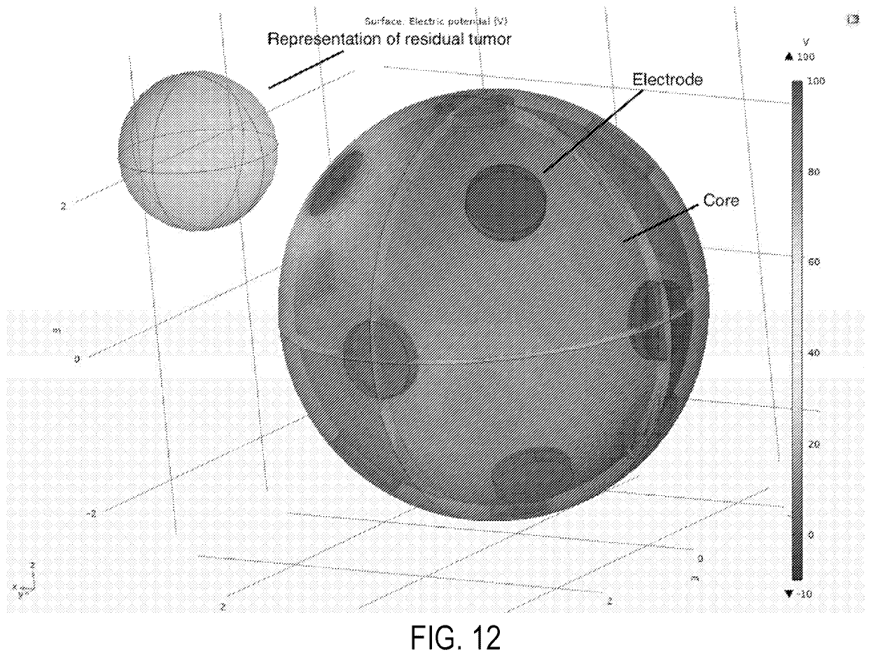

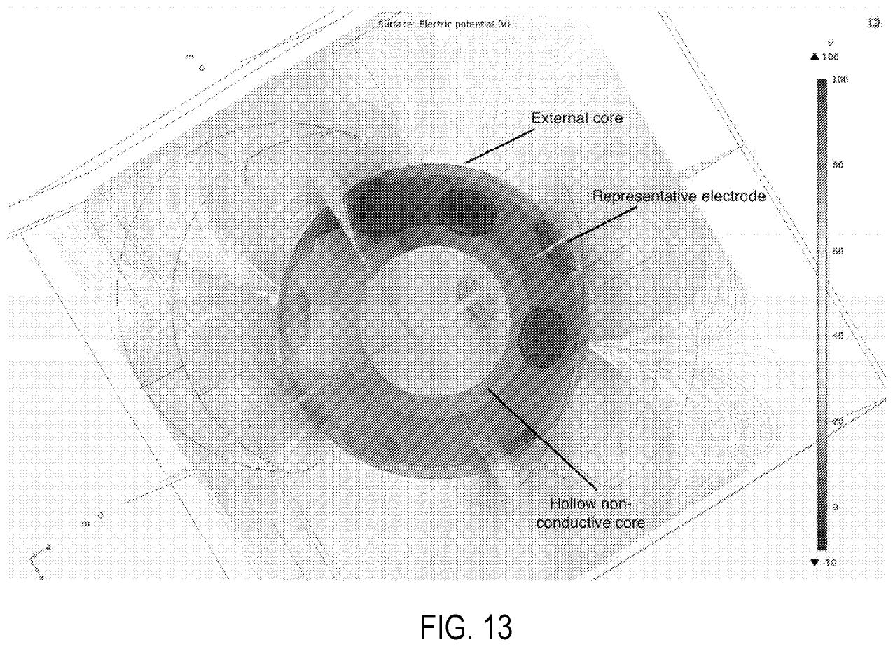

FIGS. 12 and 13 illustrate an example implementation using intra-cavitary electrode arrays. As shown in the perspective view of FIG. 12, an example of an electrode array in accordance with the present disclosure. The electrode array includes a core, which may be formed of a non-conductive material. As shown in FIG. 13, the core may be a hollow shell. On an outer surface of the core, a plurality of electrodes are disposed. In the implementation illustrated in FIG. 12, the electrodes are evenly spaced from one another; however, in other implementations the electrode spacing may be variable. A current source and/or current sink may be disposed within the core in some implementations. Upon applying a potential difference to a set of electrodes (e.g., a pair of electrodes), current flows through the peri-tumoral tissue (e.g., through the residual tumor) due to the non-conductivity of the core material. FIG. 13 shows the resulting electric potential in the external core of the device, with field lines in the surrounding tissue.

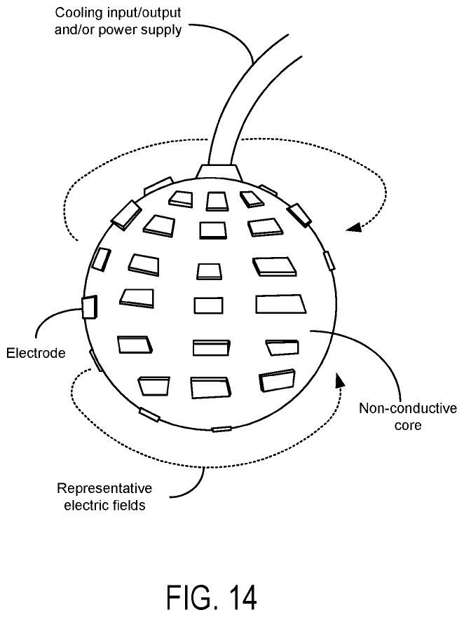

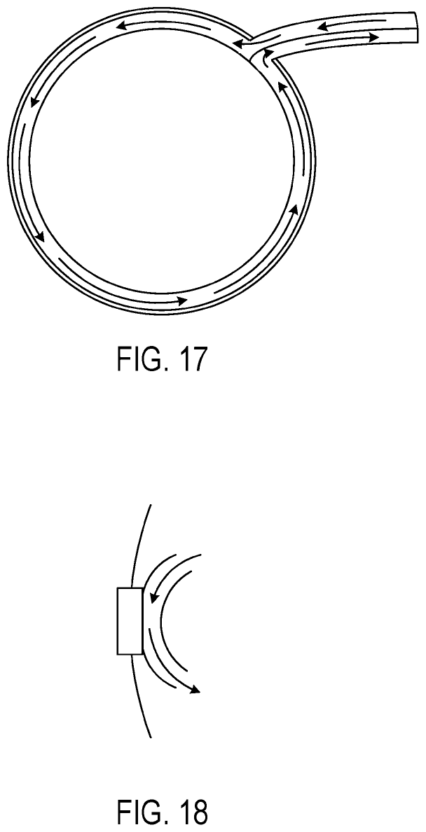

FIG. 14 illustrates another example implementation of an intra-cavity electrode array. Similar to FIG. 13, the implementation shown in FIG. 14 includes a non-conductive core and an array of electrodes disposed on the surface thereof. FIG. 14 further illustrates an external conduit. In implementations where the intra-cavity electrode array does not include an internal power supply, the external conduit may be connected to an external power supply. An “external” power supply may be an implantable power supply that is external to the electrode array, or a power supply located outside the cavity such that the external conduit connects the intracavity space with an exterior space. In addition or alternatively, the external conduit may provide a cooling input/output, as will be discussed in more detail below with regard to FIGS. 17 and 18. FIG. 14 further illustrates representative electric field lines (dotted lines) which may result from sending current through certain electrodes.

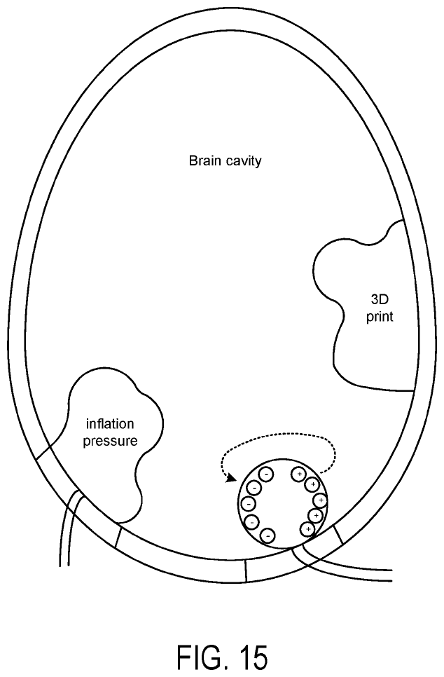

FIG. 15 illustrates three examples of an intra-cavity device disposed in a cavity. In the particular implementation illustrated, the cavity is a brain cavity; however, as noted above, the present disclosure is not limited to intracranial electrode arrays. In other implementations, the cavity may be any cavity where TTF treatment is sought. FIG. 15 illustrates an electrode array being disposed through a first section of the cranium that has been removed by, for example, a craniotomy, an expansile balloon-like array being disposed through a second section of the cranium that has been removed, and a 3D printed device coated with electrodes. The electrode array is illustrated as being connected to a power supply and/or cooling system by an external conduit, similar to that shown in FIG. 14. The electrode array is illustrated as circular in cross-section (and thus, e.g., spherical or cylindrical); however, in other implementations the electrode array may have a different three-dimensional shape.

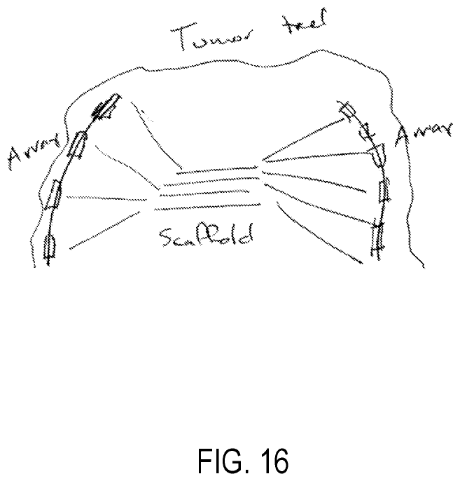

FIG. 16 illustrates an example of a scaffolding configuration, in which an interior member provides structural support for the electrodes.

As noted above, the intra-cavity electrode array implementation may be cooled, for example to mitigate the effects of heat generated by the operation of the electrodes. FIG. 17 illustrates a passive cooling example, and FIG. 18 illustrates an active cooling example. In FIG. 17, a cross-section of an electrode array (e.g., the electrode array of FIG. 14) is shown. An outer shell of the core includes a plurality of electrodes, and a coolant passageway is disposed underneath the outer shell of the core. The coolant passageway defines a conduit through which a coolant can flow, as illustrated by the arrows in FIG. 17. The coolant may be, for example, water, ethanol, and the like, and may flow from a heat sink, around the surface of the intra-cavity electrode, and back to the heat sink.

In an active cooling implementation, the cooling may be either open loop or closed loop, and may be coupled (e.g., a single cooling loop for multiple electrodes in the array) or individual (e.g., a single cooling loop for each electrode in the array). FIG. 18 illustrates an electrode at a surface of the electrode array, only a portion of which is shown. Beneath the electrode, a coolant flows as indicated by the arrows in FIG. 18. The coolant passageway may be connected to the electrode by an active device, such as a Peltier junction.

Other examples and uses of the disclosed technology will be apparent to those having ordinary skill in the art upon consideration of the specification and practice of the invention disclosed herein. The specification and examples given should be considered exemplary only, and it is contemplated that the appended claims will cover any other such embodiments or modifications as fall within the true scope of the invention.

The Abstract accompanying this specification is provided to enable the United States Patent and Trademark Office and the public generally to determine quickly from a cursory inspection the nature and gist of the technical disclosure and in no way intended for defining, determining, or limiting the present invention or any of its embodiments.

What is claimed is:

1. An intracavity device, comprising:
a non-conductive core configured to be implanted in a body surface; and
a plurality of electrodes disposed on an outer surface of the non-conductive core,
wherein the plurality of electrodes are configured to generate a tumor treating field within the body surface.

2. The intracavity device according to claim 1, wherein the non-conductive core is hollow.

3. The intracavity device according to claim 1, further comprising a cooling device disposed within the non-conductive core, wherein the cooling device includes a coolant pathway connected to a heat sink through which a coolant is configured to flow.

4. The intracavity device according to claim 1, further comprising a cooling device disposed within the non-conductive core, wherein the cooling device includes an active heat transfer element in communication with a heat sink.

5. The intracavity device according to claim 1, wherein the non-conductive core is formed of an expansile material.

6. The intracavity device according to claim 1, wherein the non-conductive core is formed of a 3D-printed material.

7. The intracavity device according to claim 1, wherein the non-conductive core includes an interior scaffold configured to support the plurality of electrodes.

8. The intracavity device according to claim 1, wherein the plurality of electrodes are evenly spaced.

9. The intracavity device according to claim 1, wherein the plurality of electrodes are configured to apply a current of less than 0.9 A.

10. The intracavity device according to claim 1, wherein respective ones of the plurality of electrodes comprise a 2×2 array of sub-electrodes.

11. The intracavity device according to claim 1, wherein the plurality of electrodes are configured to apply an electric field to inhibit or arrest proliferation of a tumor cell located in a proximity of the intracavity device.

12. The intracavity device according to claim 11, wherein the electric field is 2.25 V/cm or greater within the tumor cell.

13. The intracavity device according to claim 1, wherein the plurality of electrodes are individually activatable.

14. The intracavity device according to claim 1, further comprising an implantable power supply connected to the plurality of electrodes.

15. The intracavity device according to claim 1, further comprising an external power supply and a power conduit, wherein the power conduit is connected to the power supply and to the plurality of electrodes.

16. The intracavity device according to claim 1, wherein the body surface is a cranium, and the intracavity device is configured to be implanted intracranially.

17. A method of operating an intracavity device, comprising:
implanting the intracavity device within a body surface, wherein the intracavity device includes a non-conductive core and a plurality of electrodes disposed on an outer surface of the non-conductive core; and
generating a tumor treating field within the body surface via the plurality of electrodes.

18. The method of claim 1, wherein the operation of implanting includes creating an opening in the body surface, and placing the intracavity device in the opening.

19. The method of claim 1, wherein the operation of generating a tumor treating field includes selectively energizing ones of the plurality of electrodes to generate an electric field in an intracavity area.

20. The method of claim 19, wherein the intracavity area is an intracranial area.
